(19) United States
(12) Patent Application Publication
(10) Pub. No.: US 2009/0012009 A1
(43) Pub. Date: Jan. 8, 2009

(54) Composition and Method for Treating Inflammatory Disease

(76) Inventors: Philip S. Low, West Lafayette, IN (US); Sumith A. Kularatne, West Lafayette, IN (US)

Correspondence Address: BARNES & THORNBURG LLP, 11 SOUTH MERIDIAN, INDIANAPOLIS, IN, 46204, US

(21) Appl. No.: 12/130,121

(22) Filed: May 30, 2008

Related U.S. Application Data
(60) Provisional application No. 60/932,823, filed on Jun. 1, 2007.
(60) Provisional application No. 60/941,840, filed on Jun. 4, 2007.

(51) Int. Cl. A61K 38/05; A61K 31/519; A61P 29/00

(52) U.S. Cl. 514/19; 514/249

(57) ABSTRACT

A method of treating inflammatory diseases, and compositions and compounds therefor are described. More particularly, a method of treating inflammatory disease states with vitamin-hapten conjugates is described.

### CROSS REFERENCE TO RELATED APPLICATIONS

This application claims priority under 35 U.S.C. § 119(e) to U.S. Provisional Application No. 60/932,823, filed Jun. 1, 2007, and to U.S. Provisional Application No. 60/941,840, filed Jun. 4, 2007, which are expressly incorporated by reference herein.

### FIELD OF THE INVENTION

The invention relates to a method of treating inflammatory diseases, and compositions and compounds therefor. More particularly, the invention relates to a method of treating inflammatory disease states with vitamin-hapten conjugates.

### BACKGROUND AND SUMMARY OF THE INVENTION

The mammalian immune system provides a means for the recognition and elimination of foreign pathogens. While the immune system normally provides a line of defense against foreign pathogens, there are many instances where the immune response itself is involved in the progression of disease. Exemplary of diseases caused or worsened by the host's own immune response are autoimmune diseases such as multiple sclerosis, lupus erythematosus, psoriasis, pulmonary fibrosis, and rheumatoid arthritis and diseases in which the immune response contributes to pathogenesis such as atherosclerosis, inflammatory diseases, osteomyelitis, ulcerative colitis, Crohn's disease, and graft versus host disease (GVHD) often resulting in organ transplant rejection. Additional exemplary disease states include fibromyalgia, osteoarthritis, sarcoidosis, systemic sclerosis, Sjögren's syndrome, inflammations of the skin (e.g., psoriasis), glomerulonephritis, proliferative retinopathy, restenosis, and chronic inflammations.

Activated inflammatory cells, such as macrophages, can contribute to the pathophysiology of disease in some instances. Activated inflammatory cells can nonspecifically engulf and kill foreign pathogens within the cells by hydrolytic and oxidative attack resulting in degradation of the pathogen. Peptides from degraded proteins can be displayed on the inflammatory cell surface where they can be recognized by T cells, and they can directly interact with antibodies on the B cell surface, resulting in T and B cell activation and further stimulation of the immune response. Inflammatory cell types that may be associated with inflammatory disease states include macrophages, monocytes, and progenitor cells, including endothelial progenitor cells.

There is a need for the development of new therapies with reduced toxicity that are efficacious for the treatment of diseases caused or worsened by inflammatory cells, for example, macrophages, monocytes, and progenitor cells, including endothelial progenitor cells.

The folate receptor (FR) is a 38 KDa GPI-anchored protein that binds the vitamin folic acid with high affinity (<1 nM). Following receptor binding, rapid endocytosis delivers the vitamin into the cell, where it is unloaded in an endosomal compartment at low pH. Importantly, covalent conjugation of small molecules, proteins, and even liposomes to folic acid does not alter the vitamin's ability to bind the folate receptor, and therefore, folate-drug conjugates can readily enter cells by receptor-mediated endocytosis.

Because most cells use an unrelated reduced folate carrier (RFC) to acquire the necessary folic acid, expression of the folate receptor is restricted to a few cell types. With the exception of kidney and placenta, normal tissues express low or nondetectable levels of FR. It has recently been reported that FR_{β}, the nonepithelial isoform of the folate receptor, is expressed on activated (but not resting) synovial macrophages. Thus, Applicants have utilized folate-linked compounds potentially capable of altering the function of inflammatory cells, to treat inflammatory cell-mediated disease states.

In one embodiment, a method of treating an inflammatory disease state is described. The method comprises the step of administering to a patient suffering from an inflammatory disease state an effective amount of a composition comprising a conjugate or complex of the general formula A_{b}-X, where the group A_{b} comprises a vitamin capable of binding to inflammatory cells and the group X comprises a trinitrophenyl. In another embodiment, the group A_{b} comprises a folate or a folate analog. In yet another embodiment, the inflammatory cell is selected from the group consisting of macrophages, monocytes, and progenitor cells, including endothelial progenitor cells.

In another embodiment, the patient is suffering from a disease state selected from the group consisting of multiple sclerosis, lupus erythematosus, psoriasis and other inflammations of the skin, pulmonary fibrosis, rheumatoid arthritis, atherosclerosis, inflammatory lesions, osteomyelitis, ulcerative colitis, Crohn's disease, organ transplant rejection, fibromyalgia, osteoarthritis, sarcoidosis, systemic sclerosis, Sjögren's syndrome, glomerulonephritis, proliferative retinopathy, restenosis, and chronic inflammation.

In another embodiment, compositions and compounds are described for treating an inflammatory disease state wherein the compound has the formula,

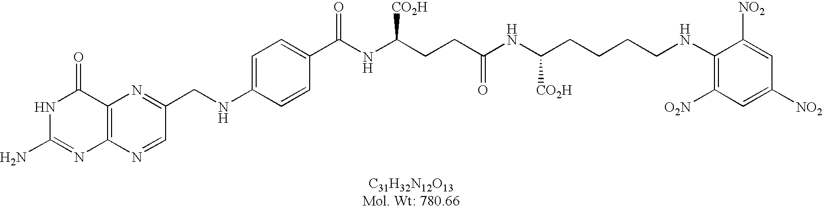

### BRIEF DESCRIPTION OF THE DRAWINGS

FIG. 1 shows a schematic representation of the synthesis of N¹⁰ TFA pteroic acid.

FIG. 2 shows a schematic representation of the synthesis of N¹⁰ TFA Folate linker for folate TNP (TriNitroPhenyl), wherein DMF is N,N-Dimethylformamide; DIPEA is N,N-Diisopropylethylamine; HOBT is 1-Hyroxybenzotriazole; TFE is Trifluoroethanol; HBTU is O-Benzotriazole-N,N,N′,N′-tetramethyl-uronium-hexafluoro-phosphate; TFA is trifluoroacetic acid; and TIPS is triisopropylsilane.

FIG. 3 shows a schematic representation of the synthesis of Folate-TNP conjugate.

FIG. 4 shows conjugates used for in vivo studies: (Panel A) structures of folate-hapten conjugates and (Panel B) schemes for their synthesis. Reagents and conditions: (i) 20% piperidine/DMF, RT, 10 min; (ii) HBTU, HOBt, DIPEA, 2 h; a: (i), Fmoc-Glu(O^{t}Bu)-OH, (ii); b: (i), Fmoc-Glu(O^{t}Bu)-OH, (ii); c: (i), N¹⁰-TFA-Ptc-OH, (ii); d: TFA/H₂O/TIPS (95:2.5:2.5), 1 h; e: aqueous NaOH (pH 10.5), 24-48 h; f: DCC, EDC/THF; g: DIPEA/DMF, RT, 12 h. RT, room temperature. Folate-DNP1=EC294; Folate-DNP2=EC293; Folate-DNP3=EC63; Folate-FITC=EC 17.

FIG. 5 shows changes in paw volumes of un-injected paws (all compounds).

FIG. 6 shows arthritis scores.

FIG. 7 shows changes in paw volumes (injected and un-injected paws).

FIG. 8 shows changes in paw volumes for injected paws (all compounds).

FIG. 9 shows changes in paw volumes for injected paws (EC63).

FIG. 10 shows changes in paw volumes for injected paws (EC293).

FIG. 11 shows changes in paw volumes for injected paws (EC294).

FIG. 12 shows changes in paw volumes for injected paws (Folate-TNP).

FIG. 13 shows changes in paw volumes for un-injected paws (EC63).

FIG. 14 shows changes in paw volumes for un-injected paws (EC293).

FIG. 15 shows changes in paw volumes for un-injected paws (EC294).

FIG. 16 shows changes in paw volumes for un-injected paws (Folate-TNP).

FIG. 17 shows changes in body weight (all compounds).

FIG. 18 shows results for gamma-scintigraphy (all compounds). Panel A: Folate-FITC (EC17); Panel B: PBS (Untreated); Panel C: folate-DNP3 (EC63); Panel D: folate-DNP2 (EC293); Panel E: folate-DNP1 (EC294); Panel F: folate-TNP; and Panel G: healthy animal.

FIG. 19 shows changes in spleen size.

FIG. 20 shows biodistribution of all compounds plotted by groups.

FIG. 21 shows biodistribution of all compounds plotted by organ.

FIG. 22 shows the content of FR+ activated macrophages in the (Panel A) spleens and (Panel B) livers of arthritic rats. The Y-axis represents the % injected dose of ^{99m}Tc-EC20 per gram tissue. Data shown are averages ±one standard deviation (n=5).

FIG. 23 shows the relative binding affinities of folate-hapten conjugates to hFR-β. CHO-β cells were incubated with 10 nM ³H-folate along with increasing concentrations (10⁻¹⁰M to 10⁻⁵M) of (∘) folic acid, (▪) folate-FITC, (▴) folate-DNP1, () folate-DNP2, (▾) folate-DNP3, and (♦) folate-TNP. Data shown are averages ±one standard deviation (n=3). Error bars are all smaller than the symbols on the graph. RBA, relative binding affinity. DPM, disintegrations per minute.

FIG. 24 shows (Panel A) timetable for immunization and treatment of animals; (Panel B) determination of antibody titers against FITC, DNP and TNP. Gray bars and open bars represent immune and pre-immune antibody titers, respectively.

FIG. 25 indicates that FR-targeted immunotherapy suppresses paw swelling and arthritis scores in rats. Arthritic rats were treated with two different doses; 30 nmol/kg (∘) or 200 nmol/kg () of each folate-hapten conjugate. Volume changes in non-injected hind paws of arthritic rats treated with (Panel A) folate-DNP1, (Panel B) folate-DNP2, (Panel C) folate-DNP3, or (Panel D) folate-TNP were measured 2×/week. Arthritis scores of all non-injected paws of rats treated with (Panel E) folate-DNP1, (Panel F) folate-DNP2, (Panel G) folate-DNP3 or (Panel H) folate-TNP were also determined 2×/week. The results of each treatment group are plotted along with the results of folate-FITC- (□) and PBS- (▪) treated rats. Data shown are averages ±one standard deviation (n=5).

FIG. 26 indicates that FR-targeted immunotherapy suppresses splenomegaly in arthritic rats. Data are presented as % change in spleen weight relative to the spleen weights of healthy rats. Data shown are averages ±one standard deviation (n=5).

FIG. 27 indicates that FR-targeted immunotherapy suppresses bone degradation in arthritic rats.

### DETAILED DESCRIPTION

Compositions, methods, and compounds are provided for the therapeutic treatment of disease states mediated by inflammatory cells. As described herein, the population of pathogenic cells cause a variety of disease states, including cancer and inflammation. Exemplary of diseases known to be mediated by inflammatory cells include rheumatoid arthritis, ulcerative colitis, Crohn's disease, psoriasis, osteomyelitis, multiple sclerosis, atherosclerosis, pulmonary fibrosis, sarcoidosis, systemic sclerosis, organ transplant rejection (GVHD) and chronic inflammations. Such disease states can be treated by administering to a patient suffering from such disease state an effective amount of a composition comprising a conjugate of the general formula A_{b}-X wherein the group A_{b} comprises a vitamin, and the group X comprises a hapten. Such conjugates, when administered to a patient suffering from inflammation, work to concentrate and associate the conjugated hapten with the population of inflammatory cells. Elimination or deactivation of the inflammatory cell population works to stop or reduce the symptoms characteristic of the disease state being treated. The conjugate is typically administered parenterally as a composition comprising the conjugate and a pharmaceutically acceptable carrier therefor. Conjugate administration is typically continued until symptoms of the disease state are reduced or eliminated.

In one embodiment, the inflammatory cells can be any inflammatory cells that cause a disease state as herein described, including but not limited to, diseases mediated by activated macrophage or activated monocytes, or other macrophage and monocyte populations that cause disease states. In one illustrative embodiment, activated macrophage mediated disease states are treated in a patient by administering a conjugate A_{b}-X wherein A_{b} comprises a vitamin and X comprises a hapten. In another illustrative embodiment, activated monocyte mediated disease states are treated in a patient by administering a conjugate A_{b}-X wherein A_{b} comprises a vitamin and X comprises a hapten.

The methods and compositions described herein can be used for both human clinical medicine and veterinary applications. In various illustrative aspects, the host animals harboring the population of pathogenic cells and treated with vitamin-hapten conjugates may be humans (e.g., a human patient) or, in the case of veterinary applications, may be laboratory, agricultural, domestic, or wild animals.

In one embodiment of the vitamin conjugates of the general formula A_{b}-X, the group A_{b} is a vitamin capable of binding to inflammatory cells, for example, activated macrophages or activated monocytes. In one embodiment, the binding ligand is a vitamin, such as folic acid, a folic acid analog or other folate receptor binding molecules. Activated macrophages express a 38 kD GPI-anchored folate receptor that binds folate and folate-derivatized compounds with subnanomolar affinity (i.e., <1 nM).

In another embodiment, the group X in the conjugate A_{b}-X, comprises a hapten, the vitamin-hapten conjugates being effective to “label” the population of inflammatory cells responsible for disease pathogenesis in the patient suffering from the disease for specific elimination by an endogenous immune response or by co-administered antibodies. In one illustrative embodiment, the use of vitamin-hapten conjugates works to enhance an immune response-mediated elimination of the inflammatory cell population. Such can be effected through an endogenous immune response or by a passive immune response effected by co-administered antibodies. The endogenous immune response may include a humoral response, a cell-mediated immune response, and any other immune response endogenous to the host animal, including complement-mediated cell lysis, antibody-dependent cell-mediated cytotoxicity (ADCC), antibody opsonization leading to phagocytosis, clustering of receptors upon antibody binding resulting in signaling of apoptosis, antiproliferation, or differentiation, and direct immune cell recognition of the delivered antigen/hapten. In another illustrative embodiment, the endogenous immune response will employ the secretion of cytokines that regulate such processes as the multiplication and migration of immune cells. The endogenous immune response may include the participation of such immune cell types as B cells, T cells, including helper and cytotoxic T cells, natural killer cells, neutrophils, LAK cells, and the like.

In another embodiment, the vitamin-hapten conjugate can be internalized and the hapten can be degraded and presented on the inflammatory cell surface, e.g. a macrophage or monocyte, for recognition by immune cells to elicit an immune response directed against macrophages presenting the degraded hapten.

Alternatively, the vitamin conjugates may be administered prophylactically to prevent the occurrence of disease in patients known to be disposed to development of inflammatory disease states. In one embodiment of the invention more than one type of vitamin conjugate can be used, for example, the host animal may be pre-immunized with fluorescein isothiocyanate and trinitrophenyl compounds and subsequently treated with fluorescein isothiocyanate and trinitrophenyl linked to the same or different targeting ligands (e.g., vitamins), in a co-dosing protocol.

The prophylactic treatment can be an initial treatment with the adjuvant and the hapten-carrier conjugate followed by treatment with the vitamin-hapten conjugate, such as treatment in a multiple dose daily regimen, and/or can be an additional treatment or series of treatments with the vitamin-hapten conjugate after an interval of days or months following the initial treatments(s) with or without administration of the adjuvant.

In one embodiment, the humoral response may be a response induced by such processes as normally scheduled vaccination, or active immunization an unnatural antigen or hapten (e.g., fluorescein isothiocyanate, a nitrophenyl, a polynitrophenyl (e.g., dinitrophenyl or trinitrophenyl), or another nitroaromatic group) with the unnatural antigen or hapten inducing a novel immunity. For example, active immunization can involve multiple injections of the unnatural antigen or hapten scheduled outside of a normal vaccination regimen to induce the novel immunity. In accordance with the methods described herein unnatural antigen, or hapten can be administered in combination with an adjuvant (in the same or different solutions), such as a quillajasaponin adjuvant (e.g., GPI-0100). In one illustrative embodiment, MHC I restricted peptides can be linked to the vitamin for use in redirecting cellular immunity to macrophages and eliciting T cell killing of macrophages.

In another illustrative embodiment, adjuvants that bias the immune response towards a T_{H}1 response can be used. In various aspects, such adjuvants can include saponin adjuvants (e.g., the quillajasaponins, including lipid-modified quillajasaponin adjuvants), CpG, 3-deacylated monophosphoryl lipid A (MPL), Bovine Calmette-Guerin (BCG), double stem-loop immunomodulating oligodeoxyribonucleotides (d-SLIM), heat-killed Brucella abortus (HKBA), heat-killed Mycobacterium vaccae (SRL172), inactivated vaccinia virus, cyclophosphamide, prolactin, thalidomide, actimid, revimid, and the like. Saponin adjuvants and methods of their preparation and use are described in detail in U.S. Pat. Nos. 5,057,540, 5,273,965, 5,443,829, 5,508,310, 5,583,112, 5,650,398, 5,977,081, 6,080,725, 6,231,859, and 6,262,029 incorporated herein by reference.

In one embodiment, the host is preimmunized with a hapten-carrier (e.g., KLH or BSA) conjugate and an adjuvant to elicit a preexisting immunity to the hapten. The vitamin-hapten conjugate is then administered to the host resulting in an humoral or cell-mediated immune response, or both, directed against the vitamin-hapten conjugate bound to the targeted inflammatory cells. In one aspect, the host is preimmunized with the hapten-carrier conjugate and the adjuvant in combination, in the same or different solutions. In this embodiment, the adjuvant enhances the immune response to the hapten upon subsequent administration of the vitamin-hapten conjugate.

In embodiments where a hapten-carrier conjugate is used, the ratio of the hapten-carrier conjugate to the adjuvant on a weight to weight basis can range from about 1:10 to about 1:1, about 1:8 to about 1:1, about 1:6 to about 1:1, about 1:4 to about 1:1, about 1:3 to about 1:1, or can be about 1:3 or about 1:2.5. In other illustrative aspects where a hapten-carrier conjugate is used, the molar ratio of the hapten-carrier conjugate to the adjuvant can range from about 1.0×10⁻³ to about 6×10⁻⁵.

In another illustrative aspect, a passive immunity may be established by administering antibodies to the host animal such as natural antibodies collected from serum or monoclonal antibodies that may or may not be genetically engineered antibodies, including humanized antibodies. The utilization of a particular amount of an antibody reagent to develop a passive immunity, and the use of a vitamin-hapten conjugate wherein the passively administered antibodies are directed to the hapten, may provide the advantage of a standard set of reagents to be used in cases where a patient's preexisting antibody titer to other potential antigens is not therapeutically useful. In one embodiment, the passively administered antibodies may be “co-administered” with the vitamin-hapten conjugate and co-administration is defined as administration of antibodies at a time prior to, at the same time as, or at a time following administration of the vitamin-hapten conjugate.

The preexisting antibodies, induced antibodies, or passively administered antibodies are redirected to the inflammatory cells by preferential binding of the vitamin-hapten conjugates to these cells. Illustratively, the pathogenic cells can be eliminated by complement-mediated lysis, ADCC, antibody-dependent phagocytosis, or antibody clustering of receptors. The cytotoxic process may also involve other types of immune responses, such as cell-mediated immunity. As used herein, the terms “eliminated” and “eliminating” in reference to the disease state, mean reducing the symptoms or eliminating the symptoms of the disease state or preventing the progression or the reoccurrence of disease. As used herein, the terms “elimination” and “deactivation” of the immune cell population that expresses the vitamin receptor mean that this cell population is killed or is completely or partially inactivated which reduces the immune cell-mediated pathogenesis characteristic of the disease state being treated.

As used herein, “mediated by” in reference to diseases mediated by inflammatory cells means caused by or augmented by. For example, inflammatory cells can directly cause disease or inflammatory cells can augment disease states such as by stimulating other immune cells to secrete factors that mediate disease states, such as by stimulating T-cells to secrete TNF-α.

In another embodiment, where there is no preexisting immunity, the vitamin-hapten conjugate, the adjuvant, and passively administered antibodies can be co-administered. In this embodiment, the passively administered antibodies help to augment the immune response to the hapten.

For all of the embodiments described herein, “co-administration” is defined as administration at a time prior to, at the same time as, or at a time following administration of the vitamin-hapten or hapten-carrier conjugate. As used herein, “co-administration” can also mean administration in the same or different solutions.

Exemplary carriers that can be used include keyhole limpet hemocyanin (KLH), haliotis tuberculata hemocyanin (HtH), inactivated diptheria toxin, inactivated tetanus toxoid, purified protein derivative (PPD) of Mycobacterium tuberculosis, bovine serum albumin (BSA), ovalbumin (OVA), g-globulins, thyroglobulin, peptide antigens, and synthetic carriers, such as poly-L-lysine, dendrimer, and liposomes.

In various illustrative embodiments, the hapten is typically conjugated to a carrier to form a hapten-carrier conjugate. The hapten and carrier can be conjugated using any method known in the art. For example, the carrier (e.g., KLH or BSA) can be conjugated to the hapten by using any art-recognized method of forming a complex including covalent, ionic, or hydrogen bonding of the carrier to the hapten, either directly or indirectly via a linking group such as a divalent linker. The hapten-carrier conjugate is typically formed by covalent bonding through the formation of amide, ester or imino bonds between acid, aldehyde, hydroxy, amino, or hydrazo groups on the respective components of the conjugates. In other embodiments, the hapten-carrier conjugate is formed by covalent bonding through the formation of bonds between hydroxy, sulfhydral guanidino or amino groups on one component and a carbon atom having a displaceable group on the other. In embodiments where a linker is used, the linker typically comprises about 1 to about 30 carbon atoms, more typically about 2 to about 20 carbon atoms. Lower molecular weight linkers (i.e., those having an approximate molecular weight of about 20 to about 500) are typically employed. In another embodiment, the linker can comprise an indirect means for associating the carrier with the hapten, such as by connection through intermediary linkers, spacer arms, or bridging molecules. Both direct and indirect means for association should not prevent the binding of the vitamin to the receptor on the cell membrane for operation of the method of the present invention.

In one illustrative embodiment, a composition comprising therapeutically effective amounts of an adjuvant and a hapten-carrier conjugate is described. In this embodiment the hapten can be fluorescein or trinitrophenyl or any other hapten. In another embodiment a composition is provided comprising therapeutically effective amounts of an adjuvant and a vitamin-hapten conjugate. A kit comprising an adjuvant, a hapten-carrier conjugate, and a vitamin-hapten conjugate is also contemplated.

In various illustrative embodiments, the vitamin-hapten conjugate may be administered to the host animal parenterally, e.g., intradermally, subcutaneously, intramuscularly, intraperitoneally, or intravenously. In other embodiments, the conjugate may be administered to the host animal by other medically useful processes, and any effective dose and suitable therapeutic dosage form, including prolonged release dosage forms, can be used. Illustratively, the method described herein may be used in combination with biological therapies such as other immunotherapies including, but not limited to, monoclonal antibody therapy, treatment with immunomodulatory agents, and vaccination.

In accordance with the methods described herein, the vitamin-hapten conjugates may be selected from a wide variety of vitamins and haptens. The vitamins can be capable of specific binding to the pathogenic cells in the host animal due to preferential expression of a receptor for the vitamin, accessible for vitamin binding, on the pathogenic cells. In various exemplary embodiments, acceptable vitamins include folic acid, analogs of folic acid and other folate receptor-binding molecules, other vitamins, and other molecules that bind specifically to a receptor preferentially expressed on the surface of activated immune cells. As used herein, “folate receptor binding ligands” includes any ligand capable of high affinity binding to the folate receptor, including folate receptor-binding analogs and derivatives.

In various embodiments, a folate receptor binding ligand can be folic acid, a folic acid analog, or another folate receptor-binding molecule. Analogs of folate that can be used include folinic acid, pteropolyglutamic acid, and folate receptor-binding pteridines such as tetrahydropterins, dihydrofolates, tetrahydrofolates, and their deaza and dideaza analogs. The terms “deaza” and “dideaza” analogs refers to the art recognized analogs having an optionally substituted carbon atom substituted for one or two nitrogen atoms in the naturally occurring folic acid structure. For example, the deaza analogs include the 1-deaza, 3-deaza, 5-deaza, 8-deaza, and 10-deaza analogs. The dideaza analogs include, for example, 1,5 dideaza, 5,10-dideaza, 8,10-dideaza, and 5,8-dideaza analogs. The foregoing folic acid analogs are conventionally termed “folates,” reflecting their capacity to bind to folate receptors. Other folate receptor-binding analogs include aminopterin, amethopterin (methotrexate), N¹⁰-methylfolate, 2-deamino-hydroxyfolate, deaza analogs such as 1-deazamethopterin or 3-deazamethopterin, and 3′,5′-dichloro-4-amino-4-deoxy-N¹⁰-methylpteroylglutamic acid (dichloromethotrexate). Any other folate receptor binding analog or derivative such as those described in U.S. Pat. Nos. 2,816,110, 5,140,104, 5,552,545, or 6,335,434, incorporated herein by reference, can also be used. Any folate analog or derivative well-known in the art, such as those described in Westerhof, et al., Mol. Pharm. 48: 459-471 (1995), incorporated herein by reference can be used.

Additional acceptable vitamins include niacin, pantothenic acid, folic acid, riboflavin, thiamine, biotin, vitamin B₁₂, and the lipid soluble vitamins A, D, E and K. These vitamins, and their receptor-binding analogs and derivatives, constitute the targeting entity that forms the vitamin-hapten conjugates as herein described. Preferred vitamin moieties include folic acid, biotin, riboflavin, thiamine, vitamin B₁₂, and receptor-binding analogs and derivatives of these vitamin molecules, and other related vitamin receptor-binding molecules (see U.S. Pat. Nos. 5,108,921, 5,416,016, and 5,635,382 incorporated herein by reference). Exemplary of a vitamin analog is a folate analog containing a glutamic acid residue in the D configuration (folic acid normally contains one glutamic acid in the L configuration linked to pteroic acid).

In one illustrative aspect, the binding site for the vitamin may include receptors for any molecule capable of specifically binding to a receptor wherein the receptor or other protein is preferentially expressed on the population of inflammatory cells, including, for example, activated immune cells.

In various illustrative aspects, the described vitamins and haptens may be conjugated by utilizing any art-recognized method of forming a conjugate, including covalent, ionic, or hydrogen bonding of the vitamin to the hapten, either directly or indirectly via a linking group such as a divalent linker. For example, the conjugate is typically formed by covalent bonding of the vitamin to the hapten through the formation of amide, ester or imino bonds between acid, aldehyde, hydroxy, amino, or hydrazo groups on the respective components of the complex. Methods of linking vitamins to haptens are described in PCT Publication No. WO 2006/012527, incorporated herein by reference.

In addition, in various embodiments structural modifications of the linker portion of the conjugates can be made. For example, a number of amino acid substitutions may be made to the linker portion of the conjugate, including but not limited to naturally occurring amino acids, as well as those available from conventional synthetic methods. In one aspect, beta, gamma, and longer chain amino acids may be used in place of one or more alpha amino acids. In another aspect, the stereochemistry of the chiral centers found in such molecules may be selected to form various mixtures of optical or stereochemical purity of the entire molecule, or only of a subset of the chiral centers present. In another aspect, the length of the peptide chain included in the linker may be shortened or lengthened, either by changing the number of amino acids included therein, or by including more or fewer beta, gamma, or longer chain amino acids. In another aspect, the selection of amino acid side chains in the peptide portion may be made to increase or decrease the relative hydrophilicity of the linker portion specifically, or of the overall molecule generally.

Similarly, the length and shape of other chemical fragments of the linkers described herein may be modified. In one aspect, the linker includes an alkylene chain. The alkylene chain may vary in length, or may include branched groups, or may include a divalent cyclic portion, which may be included in the linker. It is appreciated that the open valences on the cyclic radical may on different carbon atoms, i.e. in line, or on the same carbon atom, i.e. spiro, relative to the alkylene chain.

In one embodiment, the vitamin is folic acid, an analog of folic acid, or any other folate-receptor binding molecule. In addition, the folate ligand is conjugated to the hapten by a procedure that utilizes trifluoroacetic anhydride to prepare γ-esters of folic acid via a pteroyl azide intermediate resulting in the synthesis of a folate ligand conjugated to the hapten only through the γ-carboxy group of the glutamic acid groups of folate, thus avoiding the formation of mixtures of a γ-conjugate and an α-conjugate. Further, the γ-conjugate binds to the folate receptor with high affinity.

In another embodiment, α-conjugates can be prepared from intermediates wherein the γ-carboxy group is selectively blocked, the α-conjugate is formed and the γ-carboxy group is subsequently deblocked using art-recognized organic synthesis protocols and procedures.

In one embodiment A_{b}-X has the formula

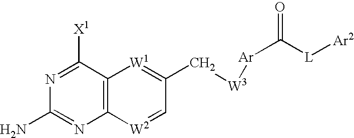

wherein X¹ is hydroxyl or amino;

W¹ and W² are each independently selected from the group consisting of N and C(R¹); where R¹ is in each instance independently selected from hydrogen, alkyl, fluoro and chloro;

W³ is O, S, N(R³) or CHR³; where R³ is hydrogen, methyl, alkyl, alkenyl, alkynyl or cyanoalkyl;

Ar is an optionally-substituted arylene;

L is a divalent linker; and

Ar² is an optionally substituted nitroaromatic group.

Illustrative examples of Ar include: 1,4-phenylene, 2,5-pyridylene, 3,6-pyridylene; 2,4-thiazolylene, 2,5-thiazolylene, 2,5-thienylene, 2,5-imidazolylene, 3,6-pyridinzylene and 2,5-pyrazinylene; each of which may be optionally substituted.

Illustrative examples of Ar² include: 4-nitrophenyl; 4-nitronaphthyl; 3,5-dinitrophenyl; 2,4,6-trinitrophenyl; and 2,4,5-trinitrophenyl.

In one embodiment, L comprises an optionally-substituted amino acid. In another embodiment, the amino acid is a naturally-occurring α-amino acid. In one embodiment L comprises a heteroatom directly bonded to Ar². In one embodiment the heteroatom is nitrogen. In another embodiment L comprises an optionally-substituted diaminoalkylene. In one embodiment the optionally-substituted diaminoalkylene is a diaminoacid. In another embodiment L comprises an optionally-substituted diaminoalkylene, and an optionally-substituted amino acid. In one illustrative example L comprises glutamic acid.

In one illustrative embodiment the hapten comprises an optionally-substituted nitroaromatic group. In one illustrative embodiment the nitroaromatic group is a polycyclic aromatic compound including one or more nitro groups. In other embodiments the nitroaromatic group is a monocyclic aromatic compound including one or more nitro groups. In one illustrative embodiment, the nitroaromatic group comprises a 3,5-dinitrophenyl fragment. In another illustrative embodiment, the nitroaromatic group comprises a 2,4-dinitrophenyl fragment. In other embodiments, the nitroaromatic group comprises a trinitrophenyl fragment. In one illustrative embodiment the nitroaromatic group is 2,4,6-trinitrophenyl.

In various embodiments, the unitary daily dosage of the vitamin-hapten conjugate can vary significantly depending on the host condition, the disease state being treated, the molecular weight of the conjugate, its route of administration and tissue distribution, and the possibility of co-usage of other therapeutic treatments such as radiation therapy. The effective amount to be administered to a patient is based on body surface area, patient weight, and physician assessment of patient condition. In various exemplary embodiments, an effective dose can range from about 1 ng/kg to about 1 mg/kg, from about 1 μg/kg to about 500 μg/kg, or from about 100 μg/kg to about 400 μg/kg (e.g., about 300 μg/kg).

Illustratively, the dosages of the adjuvant and the hapten-carrier conjugate can vary depending on the host condition, the disease state being treated, the molecular weight of the conjugate, route of administration and tissue distribution, and the possibility of co-usage of other therapeutic treatments. The effective amounts to be administered to a patient are based on body surface area, patient weight, and physician assessment of patient condition. In one illustrative aspect, effective doses of the adjuvant can range from about 0.01 μg to about 100 mg per dose, or from about 100 μg to about 50 mg per dose, or from about 500 μg to about 10 mg per dose or from about 1 mg to 10 mg per dose. In one embodiment, effective doses of the hapten-carrier conjugate can range from about 1 μg to about 100 mg per dose, or from about 10 μg to about 50 mg per dose, or from about 50 μg to about 10 mg per dose or from about 0.5 mg to about 5 mg per dose (e.g., about 3 mg per dose).

Any effective regimen for administering the adjuvant, and the hapten-carrier conjugate can be used. For example, the adjuvant and the hapten-carrier conjugate can be administered as single doses, or they can be divided (i.e., fractionated) and administered as a multiple-dose daily regimen. Further, a staggered regimen, for example, one to five days per week can be used as an alternative to daily treatment.

In exemplary embodiments, the vitamin-hapten conjugate and therapeutic factor can be administered as single doses, or they can be divided and administered as a multiple-dose daily regimen. Further, a staggered regimen, for example, one to six days per week can be used as an alternative to daily treatment. In one embodiment, the host is treated with multiple injections of the vitamin-hapten conjugate to eliminate the population of inflammatory cells. In one embodiment, the host is injected multiple times (e.g., about 2 up to about 50 times) with the vitamin-hapten conjugate, for example, at 12-72 hour intervals or at 48-72 hour intervals. Additional injections of the vitamin-hapten conjugate can be administered to the patient at an interval of days or months after the initial injections(s) and the additional injections prevent recurrence of disease. Alternatively, the initial injection(s) of the vitamin-hapten conjugate may prevent recurrence of disease.

In one embodiment, a method is provided of treating a host animal to eliminate inflammatory cells. The method comprises the steps of administering to the host animal a hapten-carrier conjugate, administering to the host animal an adjuvant wherein the ratio of the hapten-carrier conjugate to the adjuvant on a weight to weight basis ranges from about 1:10 to about 1:1, and administering to the host animal a vitamin conjugated to the hapten wherein the administration of the vitamin-hapten conjugate is initiated during the first cycle of therapy with the hapten-carrier conjugate. Illustratively, this method can be used to reduce the probability of occurrence of adverse reactions (e.g., rashes, itching, flushing). As used herein, “the first cycle of therapy” means the first, second, third, or fourth week of administration of the hapten-carrier conjugate whether or not the administration of the hapten-carrier conjugate is continuous during the first cycle of therapy.

Illustratively, in this embodiment, the pathogenic cells can be activated immune cells, such as macrophages or monocytes. In one embodiment, administration of the vitamin-hapten conjugate is initiated during the first week of therapy with the hapten-carrier conjugate. In another embodiment, administration of the vitamin-hapten conjugate is initiated during the second week of therapy with the hapten-carrier conjugate. In other embodiments, the vitamin-hapten conjugate can be administered at the start of any week of administration of the hapten-carrier conjugate as long as the administration of the vitamin-hapten conjugate is initiated before the first cycle of therapy with the hapten-carrier conjugate is complete. In various embodiments, other therapeutic factors, can be administered along with the vitamin-hapten conjugates. In another embodiment, the vitamin-hapten conjugate dose (e.g., 0.3 mg/kg (qd×5)) can be fractionated and the vitamin-hapten conjugate can be administered as fractionated doses on a daily basis (e.g., 60%, 30%, and 10% of the 0.3 mg/kg dose).

In various illustrative embodiments, the ratio of the hapten-carrier conjugate to the adjuvant on a weight to weight basis ranges from about 1:8 to about 1:1, about 1:6 to about 1:1, about 1:4 to about 1:1, about 1:3 to about 1:1, or is about 1:3 or about 1:2.5 (e.g., 1.2 mg to 3 mg per day). In one embodiment, the hapten-carrier conjugate and the adjuvant can be mixed at a weight to weight ratio of about 1:3 or about 1:2.5 or about 1:2 within about 5 minutes to about 1 hour of administration to the patient to avoid micelle formation.

Illustratively, the compositions and compounds as herein described, can be injected parenterally and such injections can be intraperitoneal injections, subcutaneous injections, intramuscular injections, intravenous injections or intrathecal injections. In another embodiment, the compositions and compounds can be delivered using a slow pump. Examples of parenteral dosage forms include aqueous solutions of the active agent in well-known pharmaceutically acceptable liquid carriers such as liquid alcohols, glycols (e.g., polyethylene glycols), glucose solutions (e.g., 5%), esters, amides, sterile water, buffered saline (including buffers like phosphate or acetate; e.g., isotonic saline). Additional exemplary components include vegetable oils, gelatin, lactose, amylose, magnesium stearate, talc, silicic acid, paraffin, and the like. In another aspect, the parenteral dosage form can be in the form of a reconstitutable lyophilizate comprising the dose of the compositions and compounds as herein described. In various aspects, solubilizing agents, local anaesthetics (e.g., lidocaine), excipients, preservatives, stabilizers, wetting agents, emulsifiers, salts, and lubricants can be used. In one aspect, any of a number of prolonged release dosage forms known in the art can be administered such as, for example, the biodegradable carbohydrate matrices described in U.S. Pat. Nos. 4,713,249; 5,266,333; and 5,417,982, the disclosures of which are incorporated herein by reference. The vitamin conjugates can also be administered topically such as in an ointment or a lotion, for example, for treatment of inflammations of the skin.

The following examples are illustrative embodiments only and are not intended to be limiting.

### EXAMPLE 1

### Materials

Heat-Killed Mycoplasma butyricum (BD Biosciences, Sparks, Md., USA); light mineral oil, bovine serum albumin, keyhole limpet hemocyanine (KLH), and alum (Sigma, St. Louis, Mo., USA); aminofluorescein (single isomer) and fluorescein isothiocyanate (FITC) (Molecular Probes, Eugene, Oreg., USA); Microcon-30 membranes (Millipore Corp., Bedford, Mass., USA); TiterMax Gold® adjuvant (CytRx Corporation, Los Angeles, Calif., USA); EC20 (a folate-linked chelator of 99mTc) and folate-FITC (Endocyte, Inc., West Lafayette, Ind., USA) were obtained from commercial sources.

### EXAMPLE 2

### Synthesis Purification and Characterization of Folate-DNP and Folate-TNP Conjugates

Picrylsulfonic acid was obtained from Wako chemicals (VA, USA) and 2,4-dinitrophenyl sulfonic acid was purchased from Avocado Research Chemicals Ltd (MA, USA). Ethyldiisopropylcarbodiimide, 2,4-dinitrophenylacetic acid and N-hydroxysuccinimide were purchased from Aldrich (MO, USA). All other chemicals were purchased from major suppliers. Compounds were purified by reverse phase preparative high performance liquid chromatography (HPLC) (Waters, xTerra C₁₈ 10 μm; 19×250 mm) and analyzed by reverse phase analytical HPLC (waters, x-bridge C₁₈ 5 μm; 3.0×15 mm). All the compounds were characterized using a Bruker 500 MHz cryoprobe NMR instrument and Waters LC-MS (ESI) mass spectrometer.

### EXAMPLE 3

### Synthesis of N¹⁰ TFA Pteroic Acid

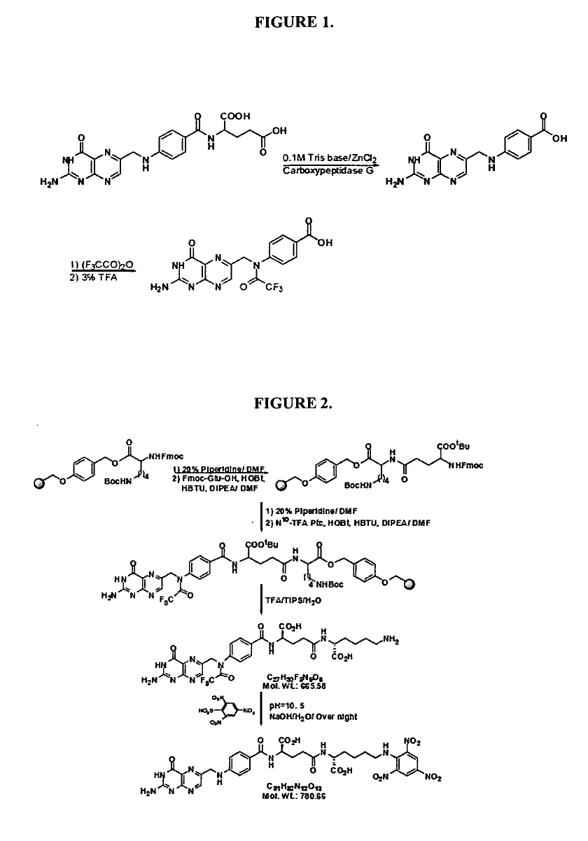

N¹⁰ TFA-Pteroic acid may be synthesized as described in PCT international application serial No. PCT/US2006/009153 (the specification of which is incorporated herein by reference), or with minor modification, as shown in FIG. 1. Briefly, zinc chloride was added to a solution of folic acid dissolved in 0.1M Tris base. Carboxypeptidase G was added to the reaction while stirring. The pH was adjusted to 7.3 using 1N HCl and the temperature was adjusted to 30° C. The reaction vessel was covered with aluminum foil and stirred for 7 days (Note: the pH and temperature must be maintained throughout the reaction). The reaction mixture was precipitated at pH 3.0 using 6N HCl and centrifuged at 4000 rpm for 10 minutes. The supernatant was decanted and lyophilized for 48 hours. The pteroic acid was purified using an ion exchange column and lyophilized for 48 hours.

The pteroic acid was dried under vacuum for 24 h and kept under argon for 30 min. Trifluoroacetic anhydride was added and stirred at room temperature under argon for 4 days (Note: the flask was wrapped with aluminum foil). Progression of the reaction was monitored by analytical HPLC [(Waters, X-Bridge C₁₈; 3.0×50 mm) and gave a single peak at λ=280 nm, 320 nm; 1% B to 50% B in 30 min, 80% B wash 35 min run]. The solvent was evaporated after the reaction was complete and 3% trifluoroacetic acid in water was added. The reaction mixture was stirred for two more days. After centrifuging at 3000 rpm for 20 minutes, the solvent was decanted and the solid was washed with water three times (Note: centrifuge and decant water each time). N¹⁰ TFA protected pteroic acid was lyophilized for 48 h.

### EXAMPLE 4

### Synthesis of N¹⁰ TFA-Folate Linker for Folate-TNP (Trinitrophenyl)

### Synthesis:

As shown in FIG. 2, folate-Lys was synthesized using standard fluorenylmethyloxycarbonyl (Fmoc) solid phase peptide synthesis (SPPS) starting from Fmoc-Lys(Boc)-Wang resin (Novabiochem; Cat. 04-12-2057). Folate-Lys linker was purified using reverse phase preparative HPLC (Waters, xTerra C₁₈ 10 μm; 19×250 mm) A=10 mM NH₄OAc (pH=7.0), B=Acetonitrile; λ=320 nm; Solvent gradient: 1% B to 70% B in 25 minutes, 80% B wash 40 minute run.

Purified compounds were analyzed using reverse phase analytical HPLC (Waters, X-Bridge C₁₈ 5 μm; 3.0×15 mm); λ=280 nm, 330 nm; 1% B to 70% B in 10 minutes, 80% B wash 15 minute run.

### Characterization:

Off-white solid, MW=665.6; C₂₇H₃₀F₃N₉O₈, Rₜ ˜ 7.8 min (analytical HPLC); LC-MS=666.4 (M+H)+; 664.3 (M−H)−; 1H NMR (Bruker 500 MHz cryoprobe, DMSO-d₆/D₂O) δ 1.23 (m, 2H, Pep-H); 1.47 (m, 3H, Pep-H); 1.63 (m, 1H, Pep-H); 1.85 (m, 1H, Pep-H); 2.08 (m, 1H, Pep-H); 2.17 (m, 2H, Pep-H); 2.71 (t, J=7.3 Hz, 2H, Pep-H); 4.01 (m, 1H, Lys-αH); 4.16 (m, 1H, Glu-αH); 5.08 (s, 2H, Ptc-H); 7.52 (d, J=8.5 Hz, 2H, Ptc-Ar—H); 7.84 (d, J=8.5 Hz, 2H, Ptc-Ar—H); 8.55 (s, 1H, Ptc-Ar—H).

### EXAMPLE 5

### Synthesis of Folate-TNP Conjugate

### Synthesis:

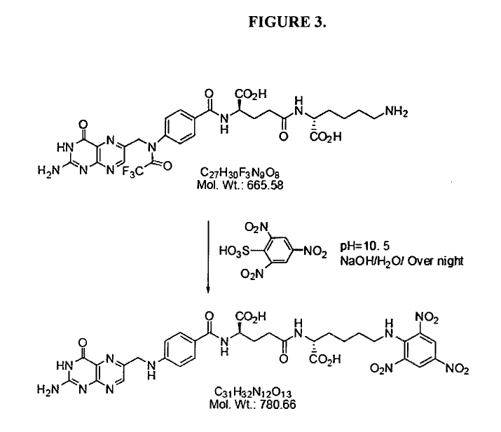

As shown in FIG. 3, folate-Lys linker was dissolved in 0.1 M NaOH solution and picrylsulfonic acid (Wako chemicals USA, Inc; catalog #209-10483) was added. The pH of the reaction mixture was adjusted to 10.5 and stirred for 48 hours. Folate TNP conjugate was purified using reverse phase preparative HPLC (Waters, XTERRA C₁₈ 10 μm; 19×250 mm) A=10 mM NH₄OAc (pH=7.0), B=Acetonitrile; λ=320 nm; Solvent gradient: 1% B to 70% B in 25 minutes, 80% B wash 40 minute run.

Purified compounds were analyzed using reverse phase analytical HPLC (Waters, X-BRIDGE C₁₈ 5 μm; 3.0×15 mm); λ=280 nm, 330 nm; 1% B to 70% B in 10 minutes, 80% B wash 15 minute run.

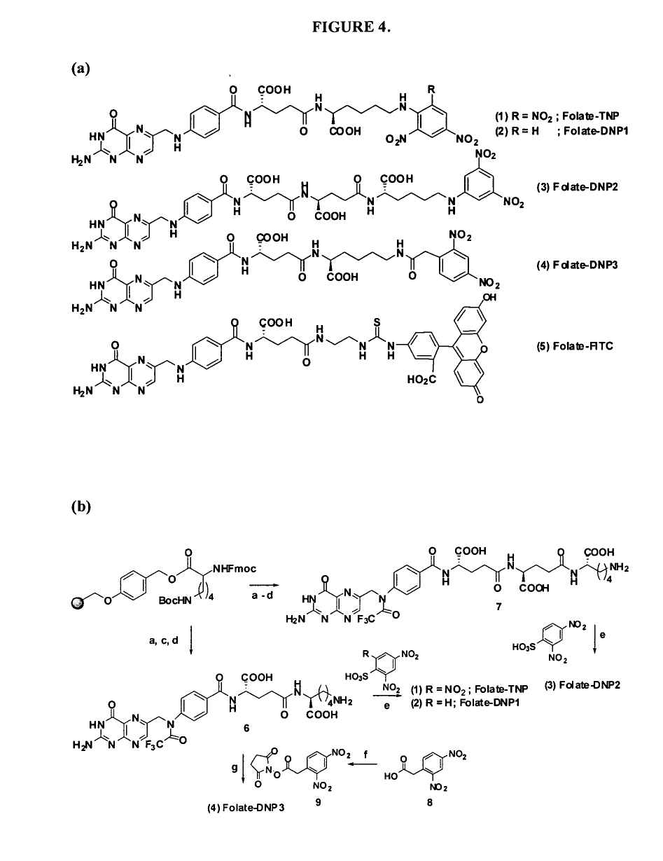

After removal of acetonitrile under reduced pressure, pure fractions were freeze-dried to yield folate-TNP as a yellow solid (FIG. 4). Rₜ ˜ 7.1 min (analytical HPLC); 1H NMR (DMSO-d₆/D₂O) δ 1.18 (m, 2H, Pep-H); 1.45 (m, 1H, Pep-H); 1.54 (m, 3H, Pep-H); 1.81 (m, 1H, Pep-H); 1.95 (m, 1H, Pep-H); 2.11 (m, 2H, Pep-H); 2.88 (m, 2H, Pep-H); 3.94 (m, 1H, Lys-αH); 4.12 (m, 1H, Glu-αH); 4.46 (s, 2H, Ptc-H); 6.61 (d, J=8.5 Hz, 2H, Ptc-Ar—H); 7.56 (d, J=8.5 Hz, 2H, Ptc-Ar—H); 8.60 (s, 1H, Ptc-Ar—H); 8.85 (s, 2H, Ar—H). LC-MS: Cal for C₃₁H₃₂N₁₂O₁₃=780.7; found=781.4 (M+H)⁺.

Folate-DNP1, folate-DNP2, and folate-DNP3 may be synthesized according to Lu et al. (2007), or with minor modifications, as shown in FIG. 4, and folate-FITC was obtained from Endocyte, Inc. (IN, USA).

### EXAMPLE 6

### Synthesis of EC63

### Synthesis:

Synthesis of EC63 was performed. 2,4-dinitrophenylacetic acid (Aldrich; Cat. 209562) was reacted with N-hydroxide succinic anhydride (NHS) in the presence of ethyldiisopropylcarbodiimide (EDC) in THF. The precipitate was filtered and washed with THF. The filtrate was concentrated under vacuum to get activated 2,4-dinitrophenylacetic acid as a solid product.

Pale brown solid, MW=323.2; C₁₂H₉N₃O₈, LC-MS=324 (M+H)+, 1H NMR (Varian 300 MHz, CDCl₃) δ 2.85 (s, 4H, CH2); 4.45 (s, 2H, CH2); 7.75 (d, J=8.4 Hz, 1H, Ar—H); 8.51 (d, J=8.4 Hz, 1H, Ar—H).

NHS activated 2,4-dinitrophenylacetic acid was dried under vacuum over night and reacted with folate-Lys linker in the presence of triethylamine (TEA) in DMF over night. The solvent was evaporated under high vacuum and water was added. The compound was freeze dried over 36 h and stirred in 1 mM NH₄HCO₃ for 1h to deprotect 10N-TFA. Final product was purified using a=10 mM NH₄HCO₃ (pH=7.8), B=Acetonitrile; λ=320 nm; Solvent gradient: 1% B to 50% B in 25 minutes, 80% B wash 40 minute run.

### Characterization:

EC 63: yellow solid, MW=777.7; C₃₃H₃₅N₁₁O₁₂; R_{t ˜}7.5 min (analytical HPLC); LC-MS=778.3 (M+H)+; 1H NMR (Bruker 500 MHz cryoprobe, DMSO-d₆/D₂O) δ 1.23 (m, 2H, Pep-H); 1.32 (m, 2H, Pep-H); 1.50 (m, 1H, Pep-H); 1.62 (m, 1H, Pep-H); 1.86 (m, 1H, Pep-H); 2.00 (m, 1H, Pep-H); 2.19 (m, 2H, Pep-H); 2.96 (m, 2H, Pep-H); 3.90 (m, 2H, CH2); 4.03 (m, 1H, Lys-αH); 4.18 (m, 1H, Glu-αH); 4.46 (s, 2H, Ptc-H); 6.61 (d, J=8.3 Hz, 2H, Ptc-Ar—H); 7.58 (d, J=8.3 Hz, 2H, Ptc-Ar—H); 7.72 (d, J=8.3 Hz, 1H, Ar—H); 8.42 (d, J=8.2 Hz, 1H, Ar—H); 8.61 (s, 1H, Ptc —Ar—H); 8.68 (s, 1H, Ar—H).

### EXAMPLE 7

### Synthesis of EC293 and EC294

### Synthesis:

EC 293 and EC 294 were synthesized and purified as described for the folate-TNP conjugate. Specifically, the corresponding folate linker was dissolved in 0.1 M NaOH solution and 2,4-dinitrophenyl sulfonic acid (Avocado Research Chemicals Ltd; Cat. 21430) was added. The pH of the reaction mixture was raised to 10.5 and stirred for 48 hours. The final products were purified A=10 mM NH₄OAc (pH=7.0), B=Acetonitrile; λ=320 nm; Solvent gradient: 1% B to 70% B in 25 minutes, 80% B wash 40 minute run. The purified compounds were analyzed using reverse phase analytical HPLC (Waters, X-Bridge C₁₈ 5 μm; 3.0×15 mm); λ=280 nm, 330 nm; 1% B to 70% B in 10 minutes, 80% B wash 15 minute run.

### Characterization:

Folate-Glu-Lys: Off-white solid, MW=794.7; C₃₂H₃₇F₃N₁₀O₁₁, Rₜ ˜7.2 min (analytical HPLC); LC-MS=795.4 (M+H)+; 1H NMR (Bruker 500 MHz cryoprobe, DMSO-d₆/D₂O) δ 1.34 (m, 2H, Pep-H); 1.54 (m, 3H, Pep-H); 1.70 (m, 2H, Pep-H); 1.84 (m, 1H, Pep-H); 2.02 (m, 1H, Pep-H); 2.12 (m, 2H, Pep-H); 2.21 (m, 2H, Pep-H); 2.26 (m, 1H, Pep-H); 2.82 (t, J=7.0 Hz, 2H, Pep-H); 4.00 (m, 1H, Lys-αH); 4.24 (m, 2H, Glu-αH); 5.14 (q, J=16.2 Hz; 2H, Ptc-H); 7.57 (d, J=8.5 Hz, 2H, Ptc-Ar—H); 7.94 (d, J=8.5 Hz, 2H, Ptc-Ar—H); 8.61 (s, 1H, Ptc-Ar—H).

EC 293: yellow solid, MW=864.7; C₂₆H₄₀N₁₂O₁₄; R_{t ˜}8.58 min (analytical HPLC); LC-MS=865(M+H)⁺; 864(M−H)−; 1H NMR (Bruker 500 MHz cryoprobe, DMSO-d₆/D₂O) δ 1.30 (m, 3H, Pep-H); 1.55 (m, 3H, Pep-H); 1.66 (m, 2H, Pep-H); 2.00 (m, 1H, Pep-H); 2.08 (m, 1H, Pep-H); 2.11 (m, 4H, Pep-H); 3.39 (t, J=7.5 Hz, 2H, Pep-H); 3.92 (m, 1H, Lys-αH); 3.97 (m, 1H, Glu-αH); 4.12 (m, 1H, Glu-αH); 4.47 (s, 2H, Ptc-H); 6.61 (d, J=8.6 Hz, 2H, Ptc-Ar—H); 7.17 (d, J=9.8 Hz, 1H, Ar—H); 7.58 (d, J=8.5 Hz, 2H, Ptc-Ar—H); 8.41 (d, J=8.7 Hz, 1H, Ar—H); 8.54 (s, 1H, Ar—H); 8.58 (s, 1H, Ptc-Ar—H).

EC 294: yellow solid, MW=735.7; C₃₁H₃₃N₁₁O₁₁; R_{t ˜}8.58 min (analytical HPLC); LC-MS=736(M+H)⁺; 734(M−H)−; 1H NMR (Bruker 500 MHz cryoprobe, DMSO-d₆/D₂O) δ 1.23 (m, 2H, Pep-H); 1.32 (m, 2H, Pep-H); 1.50 (m, 1H, Pep-H); 1.62 (m, 1H, Pep-H); 1.86 (m, 1H, Pep-H); 2.00 (m, 1H, Pep-H); 2.19 (m, 2H, Pep-H); 2.96 (m, 2H, Pep-H); 3.90 (m, 2H, CH2); 4.03 (m, 1H, Lys-αH); 4.18 (m, 1H, Glu-αH); 4.46 (s, 2H, Ptc-H); 6.61 (d, J=8.3 Hz, 2H, Ptc-Ar—H); 7.58 (d, J=8.3 Hz, 2H, Ptc-Ar—H); 7.72 (d, J=8.3, 1H, Ar—H); 8.42 (d, J=8.2, 1H, Ar—H); 8.61 (s, 1H, Ptc —Ar—H); 8.68 (s, 1H, Ar—H).

### EXAMPLE 8

### Induction and Monitoring of Experimental Arthritis in Rodents

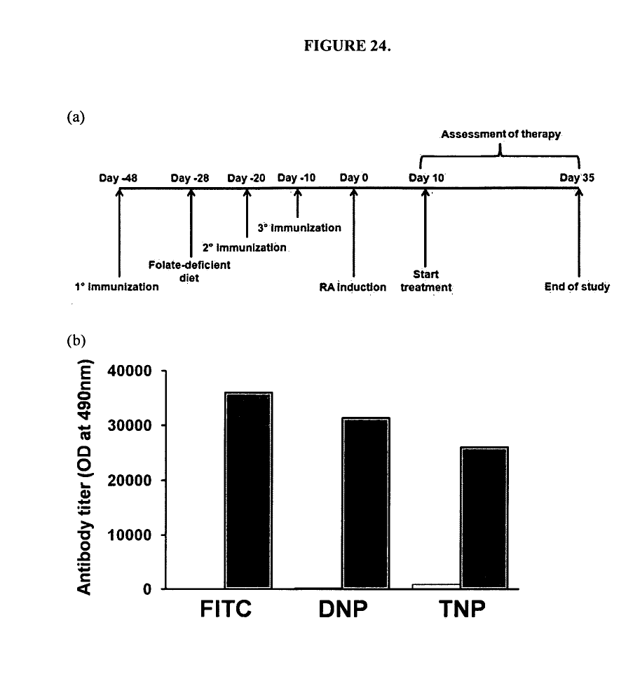

Female Lewis rats (175-200 g) were purchased from Harlan (IN, USA). All animal care and use was performed according to NIH guidelines and in compliance with protocols approved by the Purdue Animal Use and Care Committee (PACUC). Rats were kept at 22° C. in a 12-h light cycle. Four weeks prior to immunization, rats were transferred to a folate-deficient rodent diet to normalize the levels of serum folate to the physiological range (Paulos et al., 2006) (FIG. 24, Panel A).

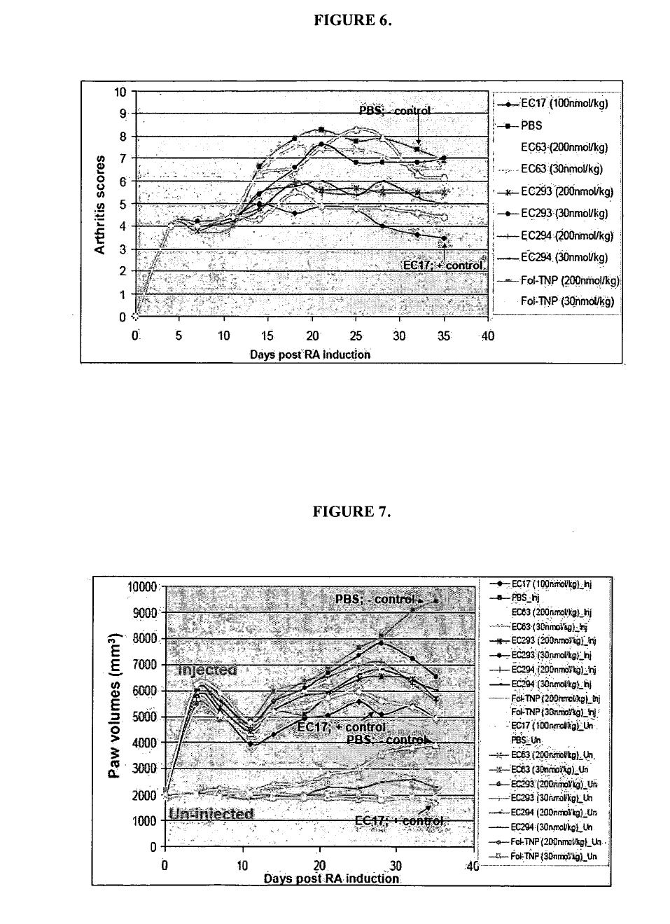

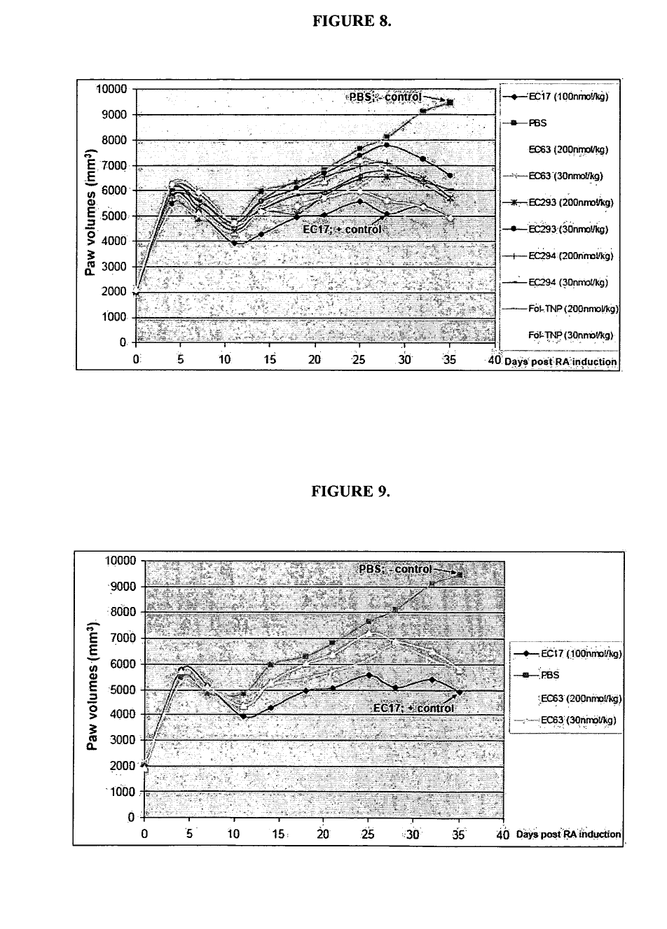

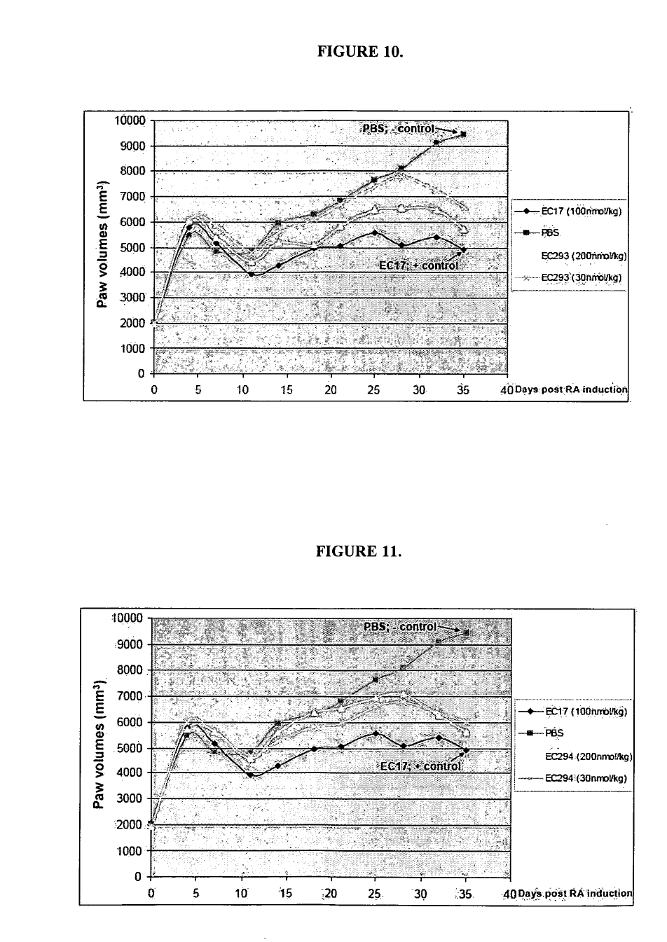

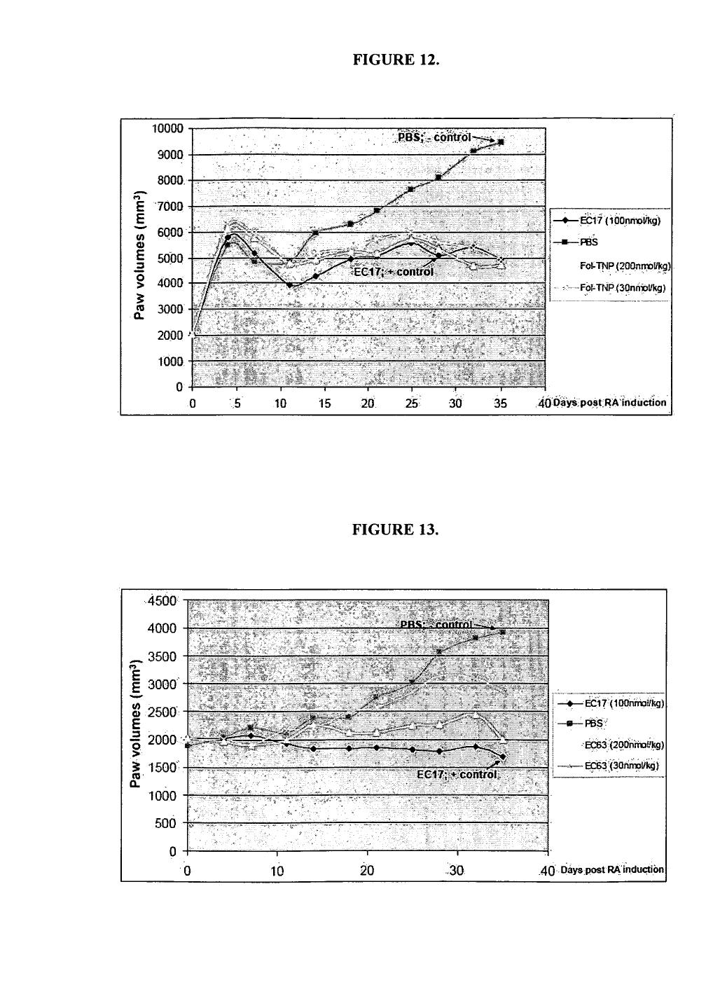

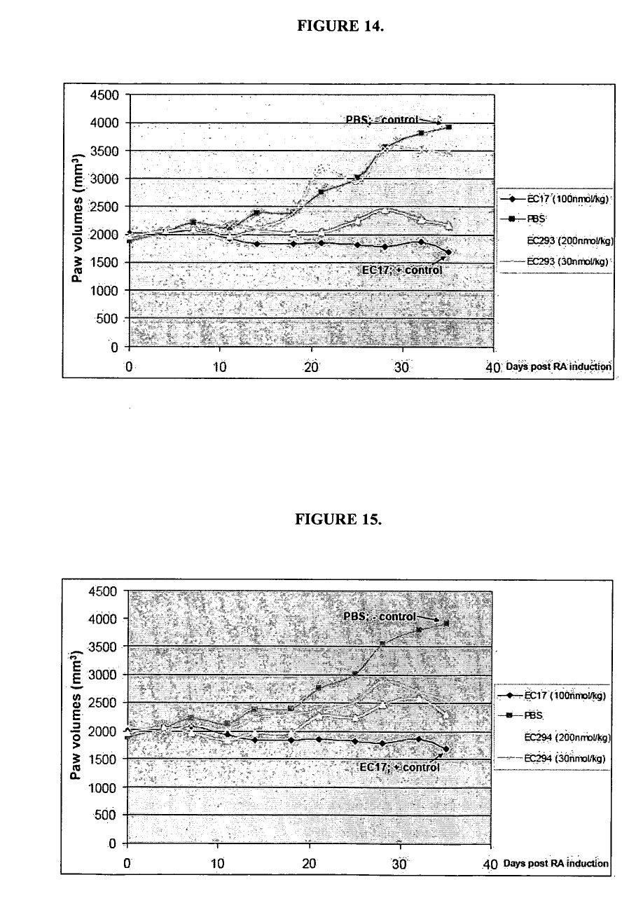

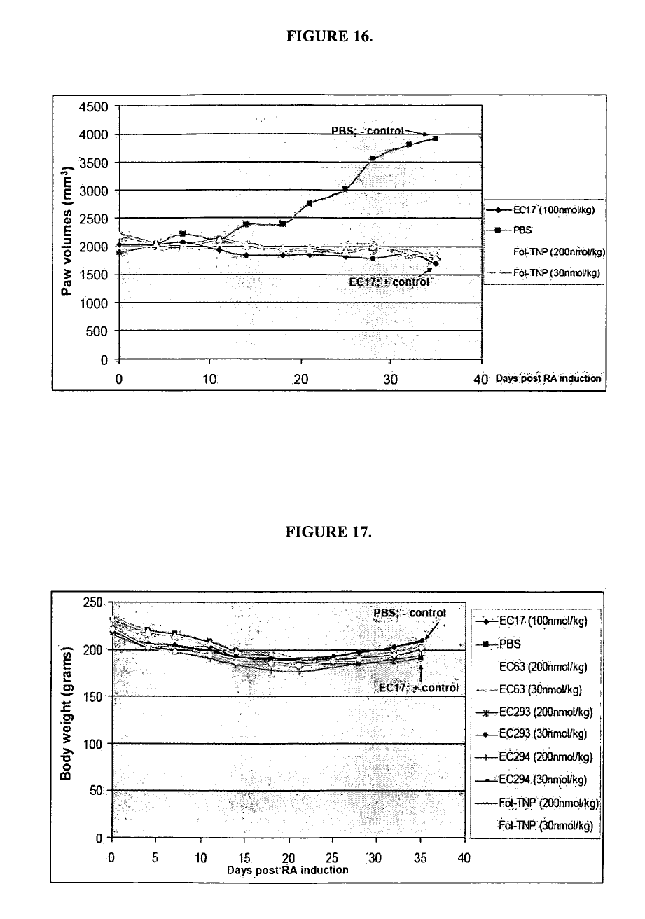

Adjuvant-induced arthritis (AIA) was promoted in 200-g female Lewis rats (Charles River Laboratories, Wilmington, Mass., USA) via either the footpad method or the base-oftail method. The arthritic rodents (rats) were weighed weekly. Total body weights are shown in FIG. 17. Arthritis scores were determined using a weighted criterion (Chondrex, Inc.) and scored by a trained investigator blinded to the treatment groups. When the arthritis score reached 7, mice were randomly assigned to different treatment groups. Rodents were maintained on a folate-deficient diet (Harlan Tec) for 3 weeks prior to each study to lower serum folate levels to their physiologic range (approximately 25 nM). See FIGS. 6-16.

### EXAMPLE 9

### Induction and Detection of Anti-Hapten Antibodies

Anti-hapten antibodies were induced in rodent models with experimental arthritis by vaccination with KLH-hapten (molar ratio of 1:13). Rodents (rats) were immunized subcutaneously with an emulsion of 150 μg KLH-hapten/200 μl adjuvant. See FIGS. 6-16.

### EXAMPLE 10

### Folate-Targeted Immunotherapy in Experimental Arthritis

Folate-hapten conjugates were administered i.p. to KLH-hapten-immunized rodents (rats) according to the doses described in each figure legend. For negative controls, KLH-hapten-immunized rodents were treated with phosphate buffered saline (PBS). See FIGS. 6-16.

### EXAMPLE 11

### Evaluation of Therapeutic Potencies

To determine whether folate-hapten conjugates could ameliorate the symptoms of experimental arthritis in rodent models, disease status was assessed by monitoring changes in limb volume/ankle diameter, radiological score (RAD score), and systemic inflammation. Limb volume was determined by calculating the product of the measured length, width, and height of the limb (average ±SD, 8 rats/group). To determine the impact of the therapies on bone/cartilage degradation, lateral radiographic projections of the tarsus of each rat were scored at the end of each study. Radiographs were taken with direct exposure (1:1) on un-screen KODAK X-OMAT TL film (Kodak, Rochester N.Y., USA) using a Faxitron X-ray system with a 0.5-mm focal spot and beryllium window (Faxitron X-ray Corporation, Wheeling, Ill., USA). Radiographs were scored by a board-certified veterinary radiologist blinded to the treatment groups. All radiographs were evaluated by a board-certified radiologist without knowledge of the assignment of treatment groups. RAD scores were assigned. The radiographic changes were graded numerically according to severity: increased soft tissue volume (0-4), narrowing or widening of joint spaces (0-5), subluxation (0-3), subchondral erosion (0-3), periosteal reaction (0-4), osteolysis (0-4), and degenerative joint changes (0-3). See FIGS. 6-16.

### EXAMPLE 12

### Scintigraphy and Biodistribution Studies

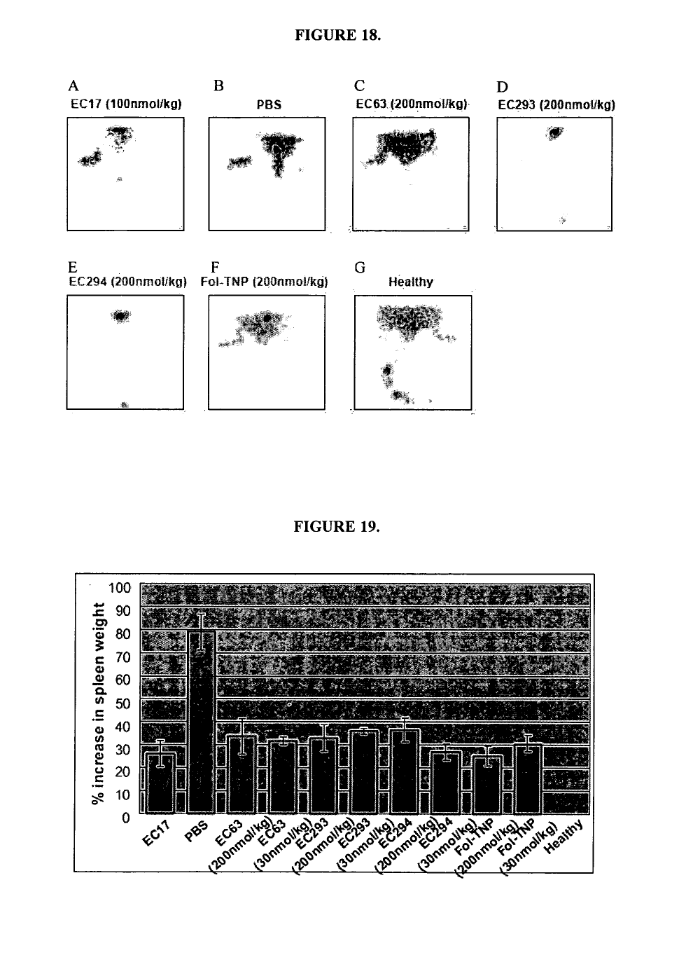

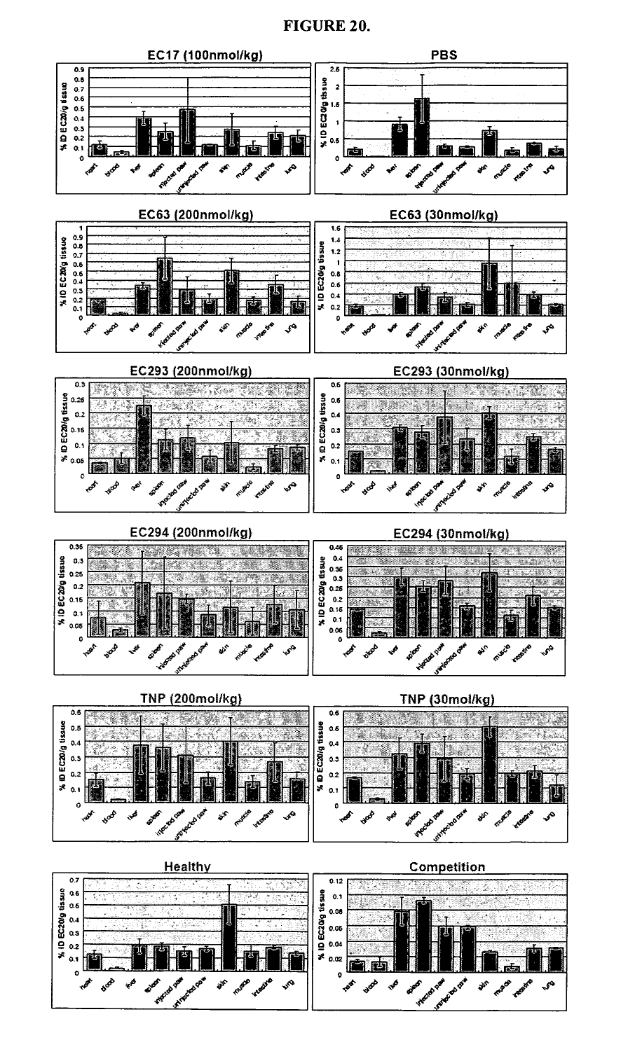

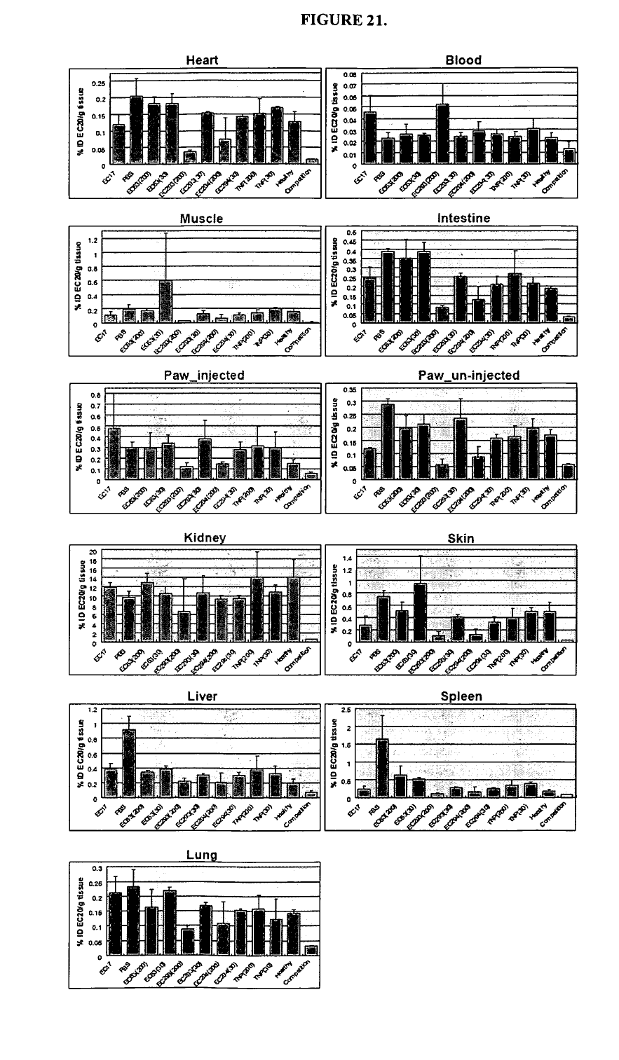

Scintigraphy and the biodistribution of folate-hapten conjugates were evaluated in relevant tissues to analyze the reduction in the number of FR+inflammatory cells. See FIGS. 18 and 20-21. FIG. 18 shows gamma-scintigraphy images of paws (lower body/kidney shielded with Pb-pad) of arthritic rats treated with a FR-targeted immunotherapeutic. Arthritic rats were treated with 200 nmol/kg of each conjugate 5×/week for 25 days and imaged with a gamma-scintigraphy imager.

### EXAMPLE 13

### Analysis of Splenomegaly

One diagnostic characteristic of systemic inflammation in adjuvant-induced arthritis is a gradual increase in spleen weight to more than twice its normal value. Therefore, to estimate the impact of the various therapies on systemic inflammation, the weight of each animals spleen was measured at the end of each study (FIG. 19).

### EXAMPLE 14

### Relative Binding Affinity Assays of Folate-DNP and Folate-TNP Conjugates to HFR-B

The relative binding affinities of folate-hapten conjugates to hFR-β were examined using a previously described method (Reddy et al., 2004). CHO-β cells expressing hFR-β were seeded on 48-well plates at 70% confluence and cultured at 37° C. in folate-deficient RPMI1640 medium (Invitrogen, CA, USA) supplemented with 1× penicillin/streptomycin (Gibco, Calif., USA) and 10% fetal bovine serum (FBS) (Atlanta Biologicals, GA, USA) in a 5% CO₂ humidified incubator. Twenty-four hours later, cells were washed twice with PBS (pH 7.4), after which a 10 nM solution of ³H-folic acid (GE Healthcare, NJ, USA) was added with increasing concentrations (10⁻¹⁰M to 10⁻⁵M) of either folate-FITC, folate-DNP1, folate-DNP2, folate-DNP3, or folate-TNP in cell culture medium. Cells were incubated at 37° C. for 1 h and washed 3× with 0.5 ml PBS. 0.5 ml of 1.0% sodium dodecyl sulfate (SDS) in PBS was added to each well, and after 5 min, cell lysates were collected and transferred to vials containing scintillation cocktail and counted for radioactivity. Relative binding affinity was defined as the molar ratio required for displacement of 50% of bound ³H-folic acid from the cell surface. Relative binding affinity of underivatized folic acid for its receptor was set as 1. Values above or below 1 represent binding affinities of compounds that are higher or lower than that of folic acid, respectively.

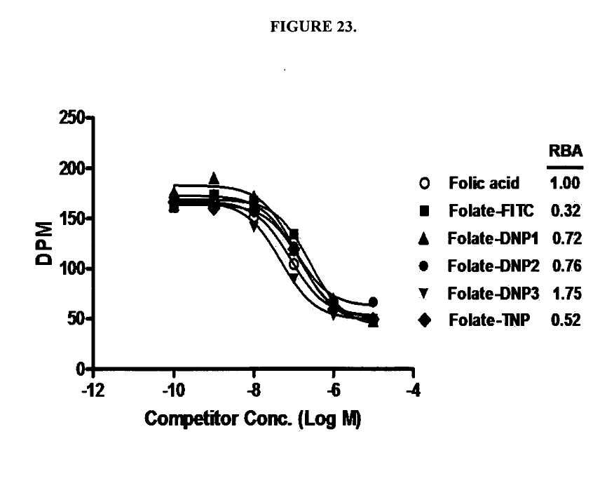

The relative binding affinities of the various folate-DNP and folate-TNP conjugates were compared by examining their association with FR-β on CHO-β cells. As shown in FIG. 23, the binding affinity of folate-DNP3 was slightly higher than that of folic acid, while those of the other folate-hapten conjugates were somewhat lower than that of folic acid. The binding affinities of all folate-DNP and folate-TNP conjugates were stronger than that of folate-FITC. The rank order of the folate-hapten conjugates was folate-DNP3>folate-DNP2>folate-DNP 1>folate-TNP>folate-FITC (FIG. 23).

### EXAMPLE 15

### Immunization and Antibody Titers

Induction of anti-hapten antibodies was achieved according to a previously described method (Paulos et al., 2006) with modifications. Rats were immunized s.c. 3× with 100 μg of either KLH-FITC, KLH-DNP (Biosearch Technologies, CA, USA), or KLH-TNP (Biosearch Technologies, CA, USA) in PBS containing GPI-0100 adjuvant (Endocyte, Inc., IN, USA) (Lu et al., 2007). Ten days after the last immunization, blood was collected by tail vein puncture, and the serum was analyzed for antibody titers against FITC, DNP, and TNP by an enzyme-linked immunosorbent assay (ELISA) (Paulos et al., 2006). Titers are presented as the dilution where 50% of each antigen is bound.

For immunotherapy of RA, a high titer of antibodies against the targeted hapten is advantageous. The titers of rats immunized with KLH-FITC, KLH-DNP or KLH-TNP are illustrated graphically in FIG. 24, Panel B. While titers were essentially similar, a weak ranking in the sequence of FITC>DNP>TNP was observed (FIG. 24, Panel B).

### EXAMPLE 16

### Induction and Observation of Experimental RA in Rats

Experimental adjuvant-induced arthritis was induced according to a previously described method (Paulos et al., 2006; van Eden et al., 1996). Briefly, adjuvant was prepared by adding finely ground heat-killed Mycobacterium butyricum(Difco Laboratories, MI, USA) in mineral oil (Sigma-Aldrich, MO, USA) at a final concentration of 1 mg/ml. The adjuvant was kept under constant stirring to ensure homogenous distribution of the mycobacterial particles. Immunized rats were anesthetized with ketamine and xylazine (100 mg/kg and 13 mg/kg, respectively) and injected in the right hind paw with 100 μl of the mycobacterial suspension. Paw inflammation was monitored daily until the first symptoms of RA appeared on the left, non-injected hind paw. Rats were randomly assigned to different treatment groups and treated as described below (FIG. 24, Panel A).

### EXAMPLE 17

### FR-Targeted Immunotherapy of Adjuvant-Induced Arthritis in Rats

To compare the efficacies of the various folate-hapten conjugates in treating RA, arthritic rats were injected i.p. 5×/week with either: 1) vehicle alone (PBS), 2) 100 nmol/kg of folate-FITC, 3) 30 nmol/kg folate-DNP1, 4) 200 nmol/kg folate-DNP1, 5) 30 nmol/kg folate-DNP2, 6) 200 nmol/kg folate-DNP2, 7) 30 nmol/kg folate-DNP3, 8) 200 nmol/kg folate-DNP3, 9) 30 nmol/kg folate-TNP, or 10) 200 nmol/kg folate-TNP. Paw volumes, arthritis scores, spleen enlargement, bone degradation, and the biodistribution of FR+macrophages were then quantitated as a function of time during therapy, as described below.

Paw volumes were measured 2×/week by multiplying length, height, and width of the non-injected hind paw (Paulos et al., 2006). Arthritis scores were graded on a scale of 0-4 2x/week by a person blinded to the treatment. Spleen enlargement was assessed 25 days after initial treatment by euthanizing the animal and measuring the weight of the resected organs. Bone degradation of the non-injected hind paw was evaluated by x-ray radiography in one representative rat from each hapten group treated at 200 nmol/kg folate-hapten conjugate. The biodistribution of FR+ macrophages in each treatment group was also quantified using the FR-targeted radioimaging agent, ^{99m}Tc-EC20, which was prepared as described previously (Turk et al., 2002). Briefly, each rat was injected i.p. with 500 μCi of radioactivity at a dose of 67 nmol/kg of EC20. Four hours later, spleens and livers were dissected, and the radioactivity of the indicated tissues was measured using a γ-scintillation counter. Relative biodistributions of ^{99m}Tc-EC20 were presented as a % injected dose per g of tissue.

Induction of experimental RA in rats

To compare the efficacies of the various folate-hapten conjugates in treating RA, experimental RA was induced in rats by injecting a heat-killed mycobacterial suspension into right hind paw (hereafter termed the injected paw) and disease symptoms were monitored in the non-injected paws. Although severe localized swelling of the injected paw was seen within one day, swelling and erythema of the non-injected paws due to systemic inflammation were first observed at˜day 10.

### Paw Volumes

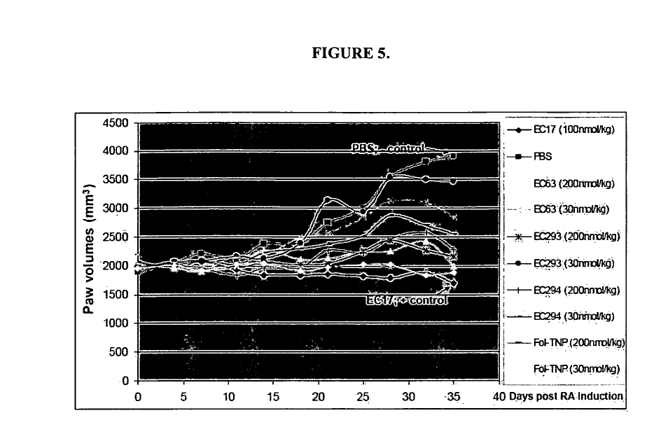

One of the diagnostic characteristics of adjuvant-induced arthritis is paw swelling (FIG. 5). To compare the potencies of the various folate-hapten conjugates in suppressing paw swelling caused by systemic inflammation, volume changes in the non-injected hind paws of arthritic rats were measured during treatment. Because previous dosing studies with folate-FITC revealed that optimal responses were observed at a daily dose of 100 nmol/kg, all dosing with the new haptens was performed at both 30 nmol/kg and 200 nmol/kg to assure that a near optimal dose was examined.

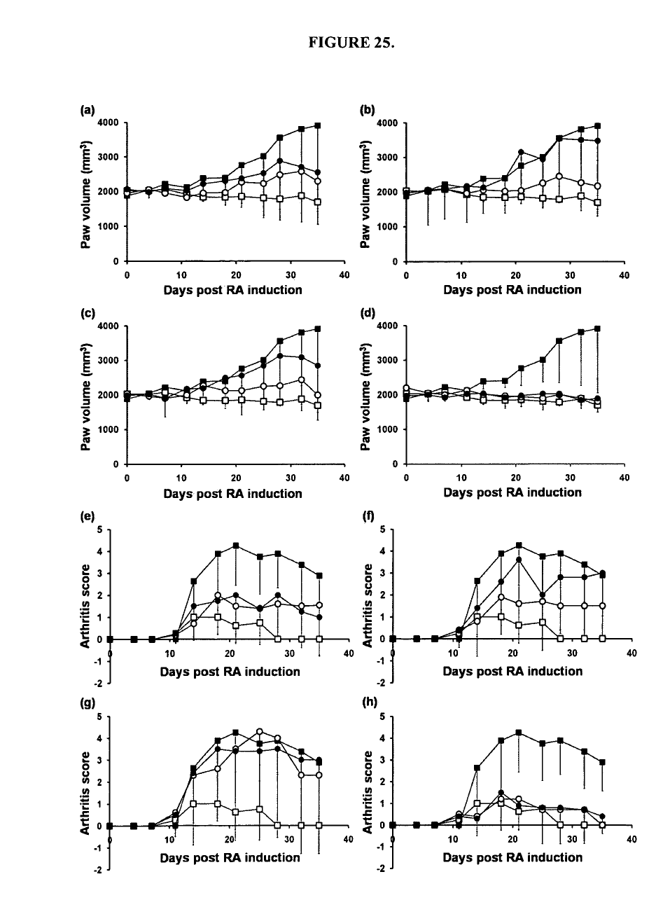

Paw swelling was observed to be reduced in all hapten-treated groups, while no reduction in paw volume was seen in the PBS-treated control (FIG. 25, Panels A-D). Folate-TNP was found to be more effective than any of the folate-DNP conjugates, but similar in potency to folate-FITC (FIG. 25, Panel D). Further, the efficacy of a 30 nmol/kg dose was similar to a 200 nmol/kg dose for folate-DNP1 and folate-TNP, but inferior to 200 nmol/kg for folate-DNP2 and folate-DNP3.

### Arthritis Scores

The relative potencies of the various haptens in preventing the increase in arthritis score characteristic of control (PBS-treated) groups was TNP=FITC>DNP1>DNP2>DNP3 (FIG. 25, Panel E-H). There did not seem to be a major impact of folate-hapten dose on arthritis score.

### Spleen Enlargement

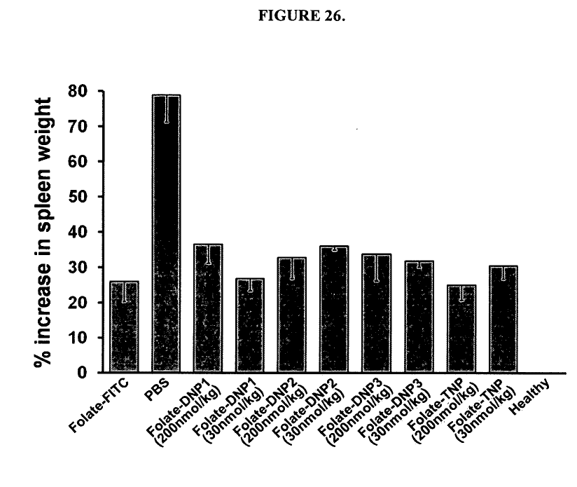

Splenomegaly, a consequence of systemic inflammation, constitutes another diagnostic characteristic of RA in both man and rats (Fletcher et al., 1998). To determine whether folate-hapten conjugates suppress splenomegaly, spleen weights were measured and compared among treatment groups. As seen in FIG. 26, immunotherapy using each of the targeted haptens led to a similar suppression of spleen enlargement. Thus, spleen weights in all hapten-treated groups increased ˜30% compared to that of healthy rats, while spleen weights in the PBS-treated group increased 80%.

### Bone Degradation

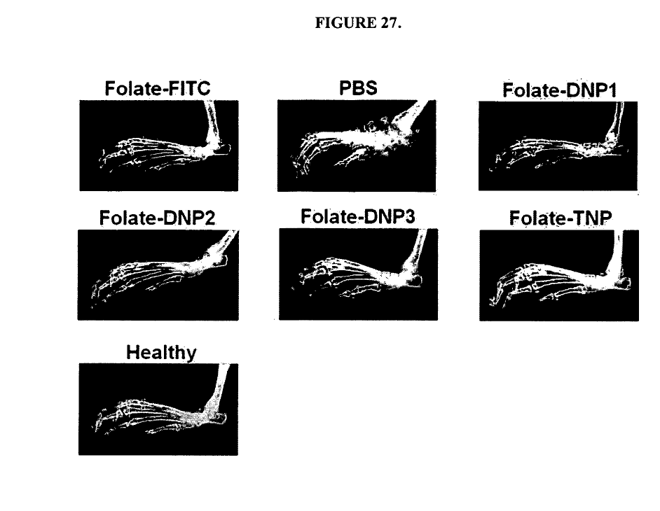

RA is also frequently characterized by progressive bone degradation. To examine whether folate-hapten conjugates suppress this bone erosion, bones of non-injected hind paws were analyzed by X-ray radiography at the end of the study. Severe bone degradation was observed in the PBS-treated group, however, bone degradation was not detectable in the groups treated with folate-hapten conjugates (FIG. 27).

Analysis of ^{99m}Tc-EC20 Biodistribution

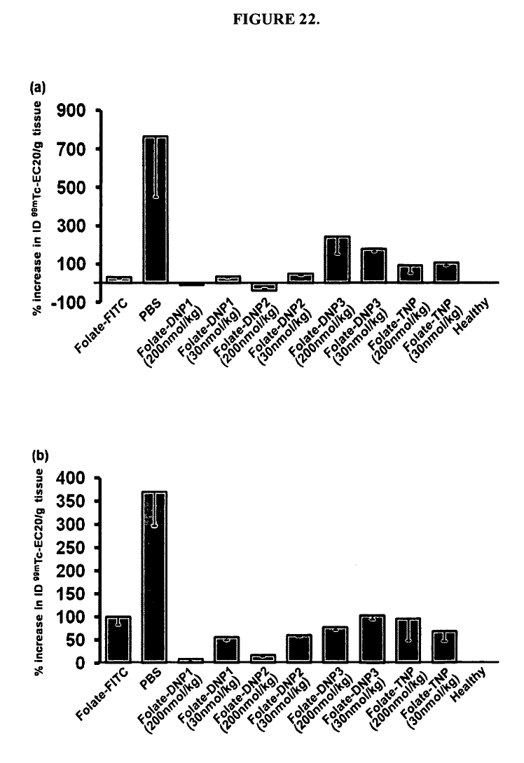

As previously reported (Paulos et al., 2006), macrophages become activated and express FR-β in the spleen, liver, and other tissues of arthritic animals. To examine whether treatment with folate-hapten conjugates depletes FR-β+activated macrophages systemically, uptake of ^{99m}Tc-EC20, a FR-targeted radioimaging agent that is internalized by FR-β+ activated macrophages, was quantitated in the above organs. High levels of ^{99m}Tc-EC20 in the spleens and livers were observed in the PBS-treated group, however, uptake of ^{99m}Tc-EC20 was markedly reduced in all groups treated with folate-hapten conjugates (FIG. 22). In this analysis, folate-DNP1 and folate-DNP2 appeared to be superior to folate-DNP3 and folate-TNP.

1. A method of treating an inflammatory disease state, said method comprising the step of administering to a patient suffering from an inflammatory disease state an effective amount of a composition comprising a conjugate or complex of the general formula
A_{b}-X
where the group A_{b} comprises a vitamin capable of binding to inflammatory cells and the group X comprises a nitroaromatic group.

2. The method of claim 1 wherein A_{b} comprises a folate receptor binding ligand.

3. The method of claim 1 wherein the inflammatory cell is selected from the group consisting of macrophages, monocytes, and progenitor cells.

4. The method of claim 3 wherein the progenitor cell is an endothelial progenitor cell.

5. The method of claim 1 wherein the patient is suffering from a disease state selected from the group consisting of multiple sclerosis, lupus erythematosus, psoriasis and other inflammations of the skin, pulmonary fibrosis, rheumatoid arthritis, atherosclerosis, inflammatory lesions, osteomyelitis, ulcerative colitis, Crohn's disease, organ transplant rejection, fibromyalgia, osteoarthritis, sarcoidosis, systemic sclerosis, Sjögren's syndrome, glomerulonephritis, proliferative retinopathy, restenosis, and chronic inflammation.

6. The method of claim 1 wherein X comprises a nitroaromatic group of formula Ar³(NO₂)ₙ wherein Ar³ is an optionally-substituted polycyclic aromatic group or an optionally-substituted monocyclic aromatic group; and n is 1 to about 4.

7. The method of claim 1 wherein X comprises trinitrophenyl.

8. The method of claim 1 wherein X comprises 2,4,6-trinitrophenyl.

9. The method of claim 1 wherein A_{b}-X is a compound of the formula 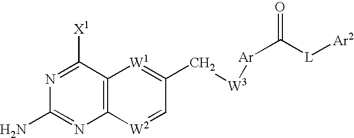
wherein X¹ is hydroxyl or amino;
W¹ and W² are each independently selected from the group consisting of N and C(R¹); where R¹ is in each instance independently selected from the group consisting of hydrogen, alkyl, fluoro and chloro;
W³ is O, S, N(R³) or CHR³; where R³ is hydrogen, methyl, alkyl, alkenyl, alkynyl or cyanoalkyl;
Ar is an optionally-substituted arylene;
L is a divalent linker; and
Ar² is an optionally substituted nitroaromatic group.

10. The method of claim 9 wherein L comprises Glu-Lys.

11. The method of claims 9 or 10 wherein Ar² is trinitrophenyl.

12. The method of claim 9 or 10 wherein A_{b}-X comprises a compound of the formula

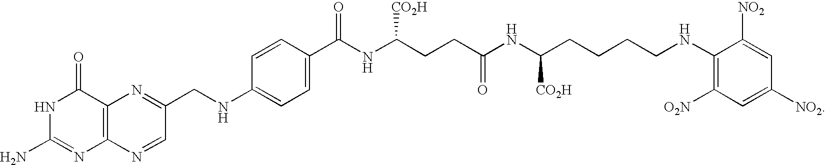

13. A compound of the formula

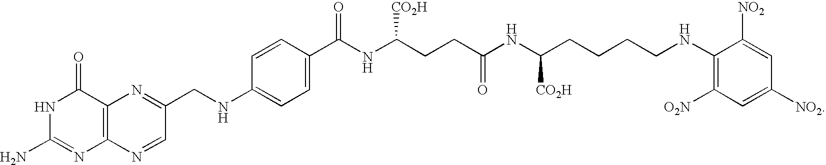

14. A method of treating an inflammatory disease state, said method comprising the step of administering to a patient suffering from an inflammatory disease state an effective amount of a composition comprising a conjugate or complex of the general formula
A_{b}-X
where the group A_{b} comprises a vitamin capable of binding to inflammatory cells and the group X comprises a nitroaromatic group of formula Ar³(NO₂)ₙ wherein Ar³ is an optionally-substituted polycyclic aromatic group or an optionally-substituted monocyclic aromatic group; and n is 1 to about 4.

15. The method of claim 14 wherein Ar³(NO₂)ₓ is 2,4,6-trinitrophenyl.

16. The method of claim 14 wherein A_{b}-X is a compound of the formula 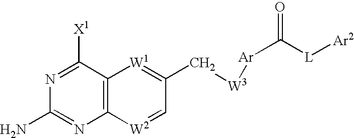
wherein X¹ is hydroxyl or amino;
W¹ and W² are each independently selected from the group consisting of N and C(R¹); where R¹ is in each instance independently selected from the group consisting of hydrogen, alkyl, fluoro and chloro;
W³ is O, S, N(R³) or CHR³; where R³ is hydrogen, methyl, alkyl, alkenyl, alkynyl or cyanoalkyl;
Ar is optionally-substituted arylene; L is a divalent linker; and
Ar² is an optionally substituted nitroaromatic group.

17. The method of claim 16 wherein Ar² is trinitrophenyl.

18. The method of claim 16 or 17 wherein L comprises Glu-Lys.
